(19) United States
(12) Patent Application Publication
(10) Pub. No.: US 2022/0018106 A1
(43) Pub. Date: Jan. 20, 2022

(54) ODOR REMOVAL SYSTEM FOR A TOILET

(71) Applicant: James Goolsby, Wortham, TX (US)

(72) Inventor: James Goolsby, Wortham, TX (US)

(21) Appl. No.: 16/932,156

(22) Filed: Jul. 17, 2020

(51) Int. Cl. E03D 9/052; A61L 9/20

(52) U.S. Cl. CPC E03D 9/052; A61L 2209/12; A61L 9/20

(57) ABSTRACT

An odor removal system disposed on a toilet, including an exhaust fan removably disposed on and within at least a portion of a bowl of the toilet to move an odor therethrough in response to a rotation of the exhaust fan, a ventilation assembly circumferentially disposed around the bowl of the toilet and connected to a first side of the exhaust fan to receive the odor from the bowl therein, and an exhaust outlet assembly connected to a second side of the exhaust fan to receive the odor extracted from the ventilation assembly by the exhaust fan and expel the odor to an outdoor environment.

### BACKGROUND

### 1. Field

The present general inventive concept relates generally to an odor removal system, and particularly, to an odor removal system for a toilet.

### 2. Description of the Related Art

Restrooms are essential for any home and/or establishment to allow a person to release bodily waste efficiently. When the person is done utilizing a toilet, the bodily waste can leave an odor even after flushing properly. Unfortunately, the odor leaves the restroom unpleasant for people arriving later.

Typically, restrooms have an exhaust fan installed in a ceiling. However, the exhaust fan relies on the odor traveling from the toilet to the ceiling. As such, people are still forced to inhale the odor until the exhaust fan in the ceiling removes the odor.

Therefore, there is a need for an odor removal system that quickly removes the odor before it leaves the toilet bowl.

### SUMMARY

The present general inventive concept provides an odor removal system for a toilet.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing an odor removal system disposed on a toilet, including an exhaust fan removably disposed on and within at least a portion of a bowl of the toilet to move an odor therethrough in response to a rotation of the exhaust fan, a ventilation assembly circumferentially disposed around the bowl of the toilet and connected to a first side of the exhaust fan to receive the odor from the bowl therein, and an exhaust outlet assembly connected to a second side of the exhaust fan to receive the odor extracted from the ventilation assembly by the exhaust fan and expel the odor to an outdoor environment.

The ventilation assembly may include a plurality of ventilation apertures disposed on a top portion of the bowl, and a ventilation chamber connected to each of the plurality of ventilation apertures to direct the odor received from the plurality of ventilation apertures toward the exhaust fan.

The exhaust outlet assembly may include an exhaust chamber connected at a first end to the exhaust fan to direct the odor from the exhaust fan toward a second end of the exhaust chamber, a check valve disposed within the exhaust chamber to rotate from closed in a first position to at least partially opened in a second position, such that the check valve regulates movement of the odor through the exhaust chamber, and an outlet vent connected to the second end of the exhaust chamber to allow the odor to move from the exhaust chamber to the outdoor environment.

The odor removal system may further include at least one sensor disposed on at least a portion of the toilet to turn on the exhaust fan in response to detecting at least one of a pressure level exceeding a predetermined pressure level on a seat of the toilet and a light level exceeding a predetermined light level around the toilet.

The at least one sensor may turn off the exhaust fan in response to detecting a water level exceeds a predetermined water level.

The odor removal system may further include a UV unit disposed on at least a portion of the toilet to eliminate the odor in response to illuminating the bowl of the toilet.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates an isometric view of an odor removal system disposed on a toilet, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Odor Removal System 100
Exhaust Fan 110
Ventilation Assembly 120
Ventilation Apertures 121
Ventilation Chamber 122
Exhaust Outlet Assembly 130
Exhaust Chamber 131
Check Valve 132
Outlet Vent 133
Sensor 140
Ultraviolet (UV) Unit 150
Power Source 160

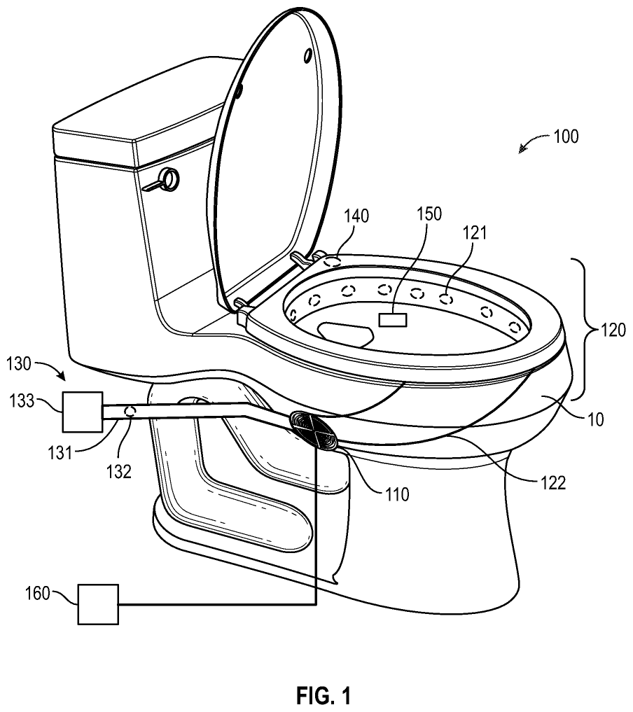

FIG. 1 illustrates an isometric view of an odor removal system 100 disposed on a toilet 10, according to an exemplary embodiment of the present general inventive concept.

The odor removal system 100 may be constructed from at least one of metal, plastic, ceramic, and rubber, etc., but is not limited thereto. Additionally, the odor removal system 100 may be highly durable and waterproof.

The odor removal system 100 may include an exhaust fan 110, a ventilation assembly 120, an exhaust outlet assembly 130, at least one sensor 140, a UV unit 150, and a power source 160, but is not limited thereto.

The exhaust fan 110 may include a fan and a vacuum, but is not limited thereto.

The exhaust fan 110 may be removably disposed on and/or within at least a portion of a bowl of the toilet 10. Moreover, the exhaust fan 110 may be disposed above a maximum water level within the bowl of the toilet 10. The exhaust fan 110 may move air and/or an odor therethrough in response to a rotation of the exhaust fan 110.

The ventilation assembly 120 may include a plurality of ventilation apertures 121 and a ventilation chamber 122, but is not limited thereto.

The plurality of ventilation apertures 121 may be circumferentially disposed around and/or within at least a portion of the bowl of the toilet 10. More specifically, the plurality of ventilation apertures 121 may be disposed on a top portion (i.e. above a water outlet portion) of the bowl of the toilet, such that water entering the bowl after flushing may not enter the plurality of ventilation apertures 121.

The plurality of ventilation apertures 121 may receive the air and/or the odor therein.

Referring to FIG. 1, the ventilation chamber 122 is illustrated to be disposed on a side of the bowl of the toilet 10. However, the ventilation chamber 122 may be circumferentially disposed around at least a portion of the bowl of the toilet 10. More specifically, the ventilation chamber 122 may connect to and envelop each of the plurality of ventilation apertures 121 to receive the air and/or the odor moving into the plurality of ventilation apertures 121. Also, the ventilation chamber 122 may be connected to a first side of the exhaust fan 110.

As such, the ventilation chamber 122 may direct the air and/or the odor toward the exhaust fan 110. In other words, the exhaust fan 110 may extract the air and/or the odor from the plurality of ventilation apertures 121 and/or the ventilation chamber 122.

The exhaust outlet assembly 130 may include an exhaust chamber 131, a check valve 132, and an outlet vent 133, but is not limited thereto.

The exhaust chamber 131 may be connected at a first end to a second side of the exhaust fan 110 and connected at a second end to the outlet vent 133. The exhaust chamber 131 may receive the air and/or the odor extracted from the ventilation chamber 122 by the exhaust fan 110 to direct the air and/or the odor toward the outlet vent 133. In other words, the exhaust fan 110 may expel (i.e. blow) the air and/or the odor into the exhaust chamber 131.

The check valve 132 may be disposed within at least a portion of the exhaust chamber 131. Moreover, the check valve 132 may rotate from closed in a first position to at least partially opened in a second position. As such, the check valve 132 may regulate movement of the air and/or the odor through the exhaust chamber 131. The check valve 132 may minimize the movement of the air and/or the odor based on its opened position. In other words, the check valve 132 may allow the air and/or the odor to move faster into the exhaust chamber 131 in a fully opened position than a partially opened position. Furthermore, the check valve 132 may create a low pressure area within the exhaust chamber 131 in response to being at least partially open, due to the exhaust chamber 131 having a smaller area for the air and/or the odor to move than an area within the bowl of the toilet 10 toward the plurality of ventilation apertures 121, the ventilation chamber 122, and/or the exhaust fan 110.

The outlet vent 133 may be connected to an outdoor environment and/or an indoor airflow system of a building. The outlet vent 133 may allow the air and/or the odor to move from the exhaust chamber 131 toward the outdoor environment and/or the indoor airflow system.

The at least one sensor 140 may include a pressure sensor, a light sensor, and a water sensor, but is not limited thereto.

Referring to FIG. 1, the at least one sensor 140 is illustrated to be disposed on at least a portion of the toilet 10, such as a seat and/or the bowl of the toilet 10. However, the at least one sensor 140 may be disposed on a wall of a restroom near the toilet 10.

The at least one sensor 140 may be electrically connected to the exhaust fan 110 and/or the check valve 132. The at least one sensor 140 may detect a pressure level on the seat, a light level in the restroom and/or around the toilet 10, and/or a water level in the toilet 10. More specifically, the exhaust fan 110 may turn on in response to a signal from the at least one sensor 140. The at least one sensor 140 may detect the pressure level on the seat of the toilet 10 exceeding a predetermined pressure level, such as when a user is disposed on the toilet 10. Alternatively, the at least one sensor 140 may detect the light level exceeding a predetermined light level, such as when a light has turned on, which indicates the user has entered the restroom. As such, the at least one sensor 140 may send the signal to the exhaust fan 110 to turn on when the user sits on the toilet 10 and/or has entered the restroom and turned on the light.

Furthermore, the at least one sensor 140 may send a stop signal to the exhaust fan 110 and/or the check valve 132 in response to detecting the water level exceeds a predetermined water level in the bowl of the toilet 10. In other words, the exhaust fan 110 may turn off and/or remain off in response to the water level reaching the exhaust fan 110, and the check valve 132 may close and/or remain closed, such that water enters the plurality of ventilation apertures 121, the ventilation chamber 122, the exhaust fan 110, and/or the exhaust chamber 131. As such, at least one sensor 140 may prevent damage to the exhaust fan 110 during an overflow of water in the toilet 10.

Alternatively, the check valve 132 may be manually manipulated by the user.

The UV unit 150 may include a UV light, but is not limited thereto.

The UV unit 150 may be disposed on and/or within at least a portion of the toilet 10. The UV unit 150 may be electrically connected to the exhaust fan 110 and/or the at least one sensor 140. The UV unit 150 may illuminate simultaneously with and/or instead of the exhaust fan 110. In other words, the UV unit 150 may operate similarly with respect to the exhaust fan 110 as described above using the at least one sensor 140. The UV unit 150 may illuminate the bowl of the toilet 10 to eliminate and/or remove the odor.

The power source 160 may be at least one of a power inlet, a battery, and a solar cell, but is not limited thereto. Additionally, the battery may include lithium-ion, nickel cadmium, nickel metal hydride, alkaline, etc., but is not limited thereto. As such, the power source 160 may charge in response to exposure to a light source and/or receiving external power from a power outlet.

The power source 160 may send power to at least one of the exhaust fan 110, the check valve 132, the at least one sensor 140, and/or the UV unit 150.

Therefore, the odor removal system 100 may eliminate the odor from the toilet 10, such that the user does not inhale the odor. Also, the odor removal system 100 may remove the odor more efficiently than a standard exhaust fan in a ceiling of the restroom.

The present general inventive concept may include an odor removal system 100 disposed on a toilet 10, including an exhaust fan 110 removably disposed on and within at least a portion of a bowl of the toilet 10 to move an odor therethrough in response to a rotation of the exhaust fan 110, a ventilation assembly 120 circumferentially disposed around the bowl of the toilet 10 and connected to a first side of the exhaust fan 110 to receive the odor from the bowl therein, and an exhaust outlet assembly 130 connected to a second side of the exhaust fan 110 to receive the odor extracted from the ventilation assembly 120 by the exhaust fan 110 and expel the odor to an outdoor environment.

The ventilation assembly 120 may include a plurality of ventilation apertures 121 disposed on a top portion of the bowl, and a ventilation chamber 122 connected to each of the plurality of ventilation apertures 121 to direct the odor received from the plurality of ventilation apertures 121 toward the exhaust fan 110.

The exhaust outlet assembly 130 may include an exhaust chamber 131 connected at a first end to the exhaust fan 110 to direct the odor from the exhaust fan 110 toward a second end of the exhaust chamber 131, a check valve 132 disposed within the exhaust chamber 131 to rotate from closed in a first position to at least partially opened in a second position, such that the check valve 132 regulates movement of the odor through the exhaust chamber 131, and an outlet vent 133 connected to the second end of the exhaust chamber 131 to allow the odor to move from the exhaust chamber 131 to the outdoor environment.

The odor removal system 100 may further include at least one sensor 140 disposed on at least a portion of the toilet 10 to turn on the exhaust fan 110 in response to detecting at least one of a pressure level exceeding a predetermined pressure level on a seat of the toilet 10 and a light level exceeding a predetermined light level around the toilet 10.

The at least one sensor 140 may turn off the exhaust fan 110 in response to detecting a water level exceeds a predetermined water level.

The odor removal system 100 may further include a UV unit 150 disposed on at least a portion of the toilet 10 to eliminate the odor in response to illuminating the bowl of the toilet 10.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An odor removal system disposed on a toilet, comprising:
an exhaust fan removably disposed on and within at least a portion of a bowl of the toilet to move an odor therethrough in response to a rotation of the exhaust fan;
a ventilation assembly circumferentially disposed around the bowl of the toilet and connected to a first side of the exhaust fan to receive the odor from the bowl therein; and
an exhaust outlet assembly connected to a second side of the exhaust fan to receive the odor extracted from the ventilation assembly by the exhaust fan and expel the odor to an outdoor environment.

2. The odor removal system of claim 1, wherein the ventilation assembly comprises:
a plurality of ventilation apertures disposed on a top portion of the bowl; and
a ventilation chamber connected to each of the plurality of ventilation apertures to direct the odor received from the plurality of ventilation apertures toward the exhaust fan.

3. The odor removal system of claim 1, wherein the exhaust outlet assembly comprises:
an exhaust chamber connected at a first end to the exhaust fan to direct the odor from the exhaust fan toward a second end of the exhaust chamber;
a check valve disposed within the exhaust chamber to rotate from closed in a first position to at least partially opened in a second position, such that the check valve regulates movement of the odor through the exhaust chamber; and
an outlet vent connected to the second end of the exhaust chamber to allow the odor to move from the exhaust chamber to the outdoor environment.

4. The odor removal system of claim 1, further comprising:
at least one sensor disposed on at least a portion of the toilet to turn on the exhaust fan in response to detecting at least one of a pressure level exceeding a predetermined pressure level on a seat of the toilet and a light level exceeding a predetermined light level around the toilet.

5. The odor removal system of claim 4, wherein the at least one sensor turns off the exhaust fan in response to detecting a water level exceeds a predetermined water level.

6. The odor removal system of claim 1, further comprising:
a UV unit disposed on at least a portion of the toilet to eliminate the odor in response to illuminating the bowl of the toilet.
